(19) United States
(12) Patent Application Publication
(10) Pub. No.: US 2013/0079755 A1
(43) Pub. Date: Mar. 28, 2013

(54) Catheter Advancement

(71) Applicant: ADAPTA MEDICAL, INC., Colorado Springs, CO (US)

(72) Inventor: Jamie Glen House, Colorado Springs, CO (US)

(73) Assignee: ADAPTA MEDICAL, INC., Colorado Springs, CO (US)

(21) Appl. No.: 13/625,840

(22) Filed: Sep. 24, 2012

Related U.S. Application Data
(60) Provisional application No. 61/538,498, filed on Sep. 23, 2011.

(51) Int. Cl. A61M 25/00

(52) U.S. Cl. CPC A61M 25/0017
USPC 604/544

(57) ABSTRACT

The present subject disclosure addresses the problems described herein by incorporating an extension in a catheter assembly for partitioning excess material of a sheath from an opening of a guide adapted to receive the catheter, thereby preventing the sheath from being pulled into the guide. Preventing the excess material from being pulled into the opening as the sheath is bunched up alleviates the difficulties of pushing the catheter through the opening.

This U.S. Patent Application claims priority to U.S. Provisional Patent Application Ser. No. 61/538,498, filed Sep. 23, 2011, the content of which is hereby incorporated by reference herein in its entirety into this disclosure.

### BACKGROUND OF THE SUBJECT DISCLOSURE

1. Field of the Subject Disclosure

The present subject disclosure relates to urinary catheters. More specifically, the present subject disclosure relates to the advancement of a catheter.

2. Background of the Subject Disclosure

Short term, or repeated catheterization of an individual's urinary bladder is a common practice today for many persons who are in a hospital setting, a nursing home, doctor's office, rehabilitation facility or at home. For instance, a user is sometimes catheterized to treat conditions such as urinary retention, the inability to evacuate urine, or for obtaining a sterile urine specimen from a user in a doctor's office.

The need for intermittent catheterization of an individual sometimes arises due to problems typically associated with long term use of indwelling catheters, such as infections, urethral damage, or bladder damage. Long term use of an indwelling catheter is also a risk factor for bladder cancer. This is often the case for persons having a neurogenic urinary condition, such as in a spinal cord injury, multiple sclerosis, stroke, trauma or other brain injury. Conditions that interfere with the individual's ability to voluntarily void the bladder may also arise post-surgically or as a result of benign prostatic hypertrophy or diabetes. Many of the affected individuals are capable of, and would prefer to perform self-catheterization. For many, the level of risk and discomfort of repeated catheterizations carried out over the course of a day (at 3-6 hour intervals, for example) are offset by the accompanying convenience, privacy or self-reliance that is achieved. Some of the major difficulties that arise in self-catheterization are the lack of satisfactory catheterization kits, the problem of maintaining the required level of sanitation during the procedure, and the difficulty of sometimes performing the procedure under conditions of restricted space and privacy.

In assisted, or non self-catheterizations, it is common practice in hospitals to employ a catheterization tray, which typically includes a sterile drape, gloves, a conventional catheter, antiseptic solution, swabs, lubricant, forceps, underpad and a urine collection container. Assisted catheterization is usually performed with the user in a supine position. Maintaining a sterile field during the procedure can still be a problem, however, and the “cath tray” procedure is impractical for use with some individuals and situations today.

Many individuals with spinal cord injuries or other neurological diseases routinely perform intermittent catheterization several times a day using conventional catheters or kits and “clean technique.” Clean technique means that the urethral area is initially swabbed with antiseptic, and efforts are made to avoid contamination of the catheter during the procedure. The user's hands are not sterile and a sterile field is not maintained. Clean technique is used instead of sterile technique, generally, for two reasons. First, it is very difficult, if not impossible, for individuals who are performing self-catheterization to adhere strictly to sterile technique. Second, these individuals are required to catheterize themselves between 3 and 6 times a day, and the cost of a new sterile catheter and the accessories required to perform sterile catheterization become excessively expensive for many users. Sometimes an individual will reuse a “cleaned” catheter. As a result, the use of non-sterile technique will many times result in contamination and subsequent infection of the urinary tract, causing significant morbidity and cost to the user and society.

Even if cost considerations were not a major consideration for the user, with most conventional self-contained sterile units where the collection bag doubles as the catheter insertion cover, the catheter is extremely difficult for the user to grasp and insert. This is particularly a problem for self-catheterization users who may also have neurological problems that limit manual dexterity. Also, with some of the available catheter kits and methods, the catheter is either not sufficiently lubricated during insertion (and thus requires the additional application of possibly non-sterile lubricant), or the catheter is too slick with lubricant and cannot effectively be grasped through an insufficiently flexible bag. As a practical matter, most individuals who would prefer to self-catheterize cannot conveniently do so, and maintain the required level of sanitation using many of the existing catheterization apparatus.

Many catheterization tasks require a degree of dexterity to accomplish. People with normal dexterity, like paraplegics, may not have use of their lower extremities, but their hands are normal. Quadriplegics can have use of their upper extremities, having absolutely normal movement, like a paraplegic, except for normal hand dexterity. Thus, many tasks requiring a degree of hand dexterity are very difficult for paraplegics to accomplish.

Spinal cord injuries at the C5, C6, or C7 level often affect the use of a person's hands and make these tasks difficult. However, people who have had strokes, brain injuries, or multiple sclerosis may also require catheterization but have limited dexterity. In this, and other ways, the current catheterization market does not currently support the needs of these people.

Advanced catheter assemblies utilize sheaths to cover the catheter, and these sheaths converge into an opening in which the catheter slides through. With the sheath having such close proximity to the opening, the sheath tends to bunch up during catheter insertion, and the friction from the advancing catheter catches and pulls the sheath into the opening. This can make it very difficult to push the catheter through the opening. pulled

### SUMMARY OF THE SUBJECT DISCLOSURE

The present subject disclosure solves the problem described above by providing an extension attached to an opening of a sheath that partitions the sheath from the opening, preventing the sheath from being pulled into the opening. Preventing the sheath from being pulled into the opening as the sheath is bunched up alleviates the difficulties of pushing the catheter through the opening.

In one exemplary embodiment, the present subject disclosure is a urinary catheter assembly. The assembly includes a catheter having a proximal end and a distal end, a guide adapted to receive the proximal end of the catheter via an opening, a sheath surrounding a substantial portion of the catheter, wherein a proximal end of the sheath is attached to the guide, and a hollow extension attached to a distal end of the guide, the hollow extension being enclosed by the sheath and being placed in between the sheath and the opening.

In another exemplary embodiment, the present subject disclosure is an extension for a urinary catheter assembly. The extension includes a hollow member, and an attachment portion at a proximal end of the hollow member, the attachment portion attachable to a distal end of a guide adapted to receive a catheter. The hollow member is in between the catheter and a sheath substantially surrounding the catheter when attached to the distal end of the guide.

In yet another exemplary embodiment, the present subject disclosure is an apparatus for a urinary catheter assembly. The apparatus includes a guide adapted to receive a proximal end of a catheter via an opening in the guide, and attachable to a proximal end of a sheath substantially surrounding the catheter, and a hollow extension coupled to a distal end of the guide. The extension is situated in between the sheath and the opening.

### BRIEF DESCRIPTION OF THE DRAWINGS

FIG. 1 shows a catheter assembly having an extension, according to an example embodiment of the present subject disclosure.

FIG. 2 shows a guide of a catheter assembly having an extension, according to an example embodiment of the present subject disclosure.

FIG. 3 shows another guide of a catheter assembly having an extension, according to an example embodiment of the present subject disclosure.

FIG. 4A shows a guide of a catheter assembly during insertion.

FIG. 4B shows the bunching of a sheath during insertion of the catheter.

FIG. 4C shows the inside of the guide during insertion of the catheter.

FIG. 5 shows a catheter assembly having an extension during insertion of the catheter, according to an example embodiment of the present subject disclosure.

FIG. 6 shows a guide and an extension as a single unit, according to an example embodiment of the present subject disclosure.

FIG. 7 shows an extension for attachment to a guide, according to an example embodiment of the present subject disclosure.

FIG. 8 shows a guide for attachment to an extension, according to an example embodiment of the present subject disclosure.

### DETAILED DESCRIPTION OF THE SUBJECT DISCLOSURE

The present application refers to subject matter described in commonly-owned U.S. Pat. No. 6,090,075, issued on Jul. 18, 2000, the contents of which are incorporated by reference herein in their entirety into this disclosure. Example embodiments of the catheter assembly include a catheter having a proximal end and a distal end. A proximal end is any portion of any part of the catheter assembly that is positioned closer to a urethra, and a distal end is any portion of any part of the catheter assembly that is positioned farther away from the urethra. The distal end of the catheter is coupled to an outlet. The catheter is enclosed in a sheath, which acts as a protective envelope for the catheter. The sheath converges at its proximal end at a guide. The guide includes an opening adapted to receive the proximal end of the catheter as it slides through the opening. Attached to the distal end of the guide is an extension that partitions the sheath from the guide. The extension may be a removable addition to the guide or manufactured as part of the guide itself. Moreover, the extension is open on both ends, such that the catheter may be inserted into the guide. Other elements may be incorporated into the catheter, and are described in U.S. Pat. No. 6,090,075.

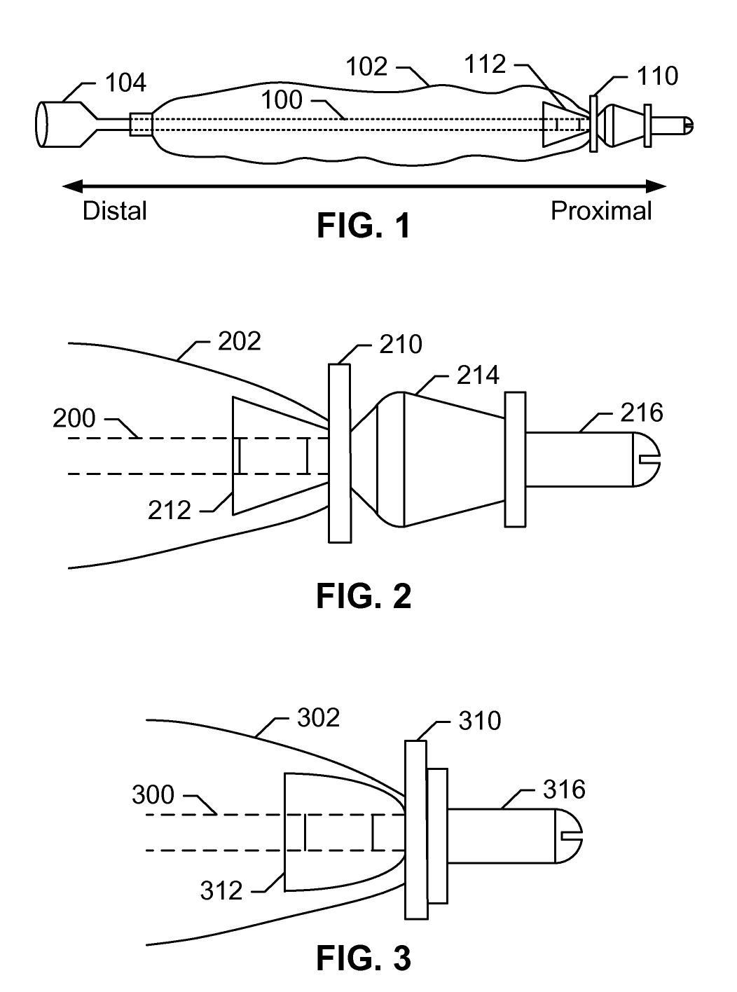

FIG. 1 shows a catheter assembly having an extension 112 according to an example embodiment of the present subject disclosure. In this example embodiment, the catheter assembly includes a catheter 100, a sheath 102, an outlet 104, a guide 110, and extension 112. A substantial portion of the length of catheter 100 is surrounded by and enclosed within sheath 102. Therefore, sheath 102 may act as a protective envelope for catheter 100. Sheath 102 converges at its proximal end at guide 110, which includes an opening adapted to receive the proximal end of catheter 100. The proximal end of catheter 100 slides through the opening in guide 110. An extension 112 is attached to the distal end of guide 110 and enclosed within sheath 102. Extension 112 serves to partition sheath 102 from the opening in guide 110, thereby preventing sheath 102 from being pulled into the opening during insertion of the catheter, as further described herein. The distal end of catheter 100 is coupled to outlet 104. Outlet 104 can be attached to a collection bag prior to insertion of the catheter or at any other time.

In this example embodiment, extension 112 is in the form of a hollow cone, which is open on both ends for purposes of catheter insertion. The proximal end of extension 112 is attached to the distal end of guide 110, the diameter of the proximal end matching or exceeding the diameter of the opening in guide 110. Extension 112 increases in diameter along its length towards the distal end of extension 112. The length of extension 112 from the proximal end to the distal end is sufficient to prevent any excess material of sheath 102 from getting pulled into the opening of guide 110 during insertion of catheter 100. As catheter 100 is being inserted into guide 110, excess material of sheath 102 bunches up close to the proximal end of the catheter assembly. Extension 112 therefore partitions this excess material of sheath 102 from the opening in guide 110, thereby preventing the excess material of sheath 102 from getting pulled into guide 110. In this manner, extension 112 can make insertion much easier.

FIG. 2 shows a guide 210 of a catheter assembly having an extension 212, according to an example embodiment of the present subject disclosure. In this example embodiment, the catheter assembly includes a catheter 200, a sheath 202, guide 210, extension 212, a reservoir 214, and an introducer 216. A substantial portion of the length of catheter 200 is surrounded by and enclosed within sheath 202, which acts as a protective envelope for catheter 200. The proximal end of catheter 200 slides through an opening in guide 210 located at the convergence of sheath 202. Attached to the distal end of guide 210 is extension 212, which partitions sheath 202 from guide 210, thereby preventing any excess material of sheath 202 from being pulled into the opening of guide 210 during insertion of the catheter. The proximal end of guide 210 is attached to reservoir 214, which contains a lubricant, water, saline, or other fluid. As the proximal end of catheter 200 enters reservoir 214 through guide 210, catheter 200 is lubricated before emerging from introducer 216 and entering the urethra. Introducer 216 is attached to the proximal end of reservoir 214. Introducer 216 is a hollow cylinder with a diameter just large enough to allow catheter 200 to slide smoothly through introducer 216 and into the urethra. Introducer 216 may be inserted into the urethra prior to insertion of the catheter into the urethra, so that catheter 200 is not exposed to the outside atmosphere and to undesired microorganisms/particles.

In this example embodiment, extension 212 of the catheter assembly is a hollow cone open on both ends for purposes of catheter insertion. The proximal end of extension 212 is attached to the distal end of guide 210, the diameter of the proximal end of extension 212 matching or exceeding the diameter of the opening in guide 210. Extension 212 increases in diameter towards the distal end of extension 212. The length of extension 212 from the proximal end to the distal end is sufficient to prevent excess material of sheath 202 from getting pulled into guide 210 during insertion of catheter 200 into the opening in guide 210. Using the extension disclosed herein, as sheath 202 starts to bunch up as catheter 200 is being inserted into the opening of guide 210, extension 212 partitions the excess material of sheath 202 from the opening of guide 210. In this manner, extension 212 prevents the excess material of sheath 202 from being pulled into the opening of guide 210, making insertion of the catheter much easier.

The extension may be made of rigid or semi-rigid transparent or translucent plastic or silicon, or any other biocompatible, sterilizable and sufficiently inflexible material, and can be the same material as the guide. In any case, the material must withstand the pressure exerted by the sheath as it tries to bunch up while the catheter is being inserted into the opening of the guide, thus achieving the objective of preventing excess material of the sheath from getting pulled into the guide.

Reservoir 214 may contain a lubricant, water, saline, or any other fluid, depending on the type of catheter 200. For many catheter types, a water-based lubricant, such as KY JELLY®, etc., may be used to lubricate catheter 200 for urethral insertion. For catheters made of a hydrophilic material or having a hydrophilic coating, reservoir 214 may contain water or saline to lubricate these catheter types. Hydrophilic catheters are further described in commonly-owned U.S. Pat. No. 8,177,774, issued on May 15, 2012, the contents of which are incorporated by reference herein in their entirety.

The extensions of the above catheter assemblies may take on different shapes and forms in different embodiments that all perform the function of partitioning the sheath from the opening, preventing excess material of the sheath from being pulled into the opening during insertion of the catheter. In another exemplary embodiment, the catheter assembly includes an extension in the form of a hollow hemisphere, which is open on both ends for purposes of catheter insertion. The proximal end of the extension is attached to the distal end of the guide, with the diameter of the proximal end matching the diameter of the opening in the guide for enabling insertion of the catheter. The diameter of the extension immediately enlarges from the proximal end, then bevels inward toward the distal end to form its hemispherical shape.

FIG. 3 shows a guide 310 of a catheter assembly having an extension 312, according to an example embodiment of the present subject disclosure. In this example embodiment, the catheter assembly includes a catheter 300, a sheath 302, guide 310, extension 312, and an introducer 316. A substantial portion of the length of catheter 300 is surrounded by and enclosed within sheath 302, which acts as a protective envelope for catheter 300, and converges at guide 310. The proximal end of catheter 300 slides through an opening in guide 310 and into introducer 316. Attached to the distal end of guide 310 is extension 312, which partitions sheath 302 from guide 310. The proximal end of guide 310 is attached to introducer 316. Introducer 316 is a hollow cylinder with a diameter just large enough to allow catheter 300 to slide smoothly through introducer 316, emerge from introducer 316 and into the urethra. Introducer 316 may enter the urethra prior to catheter entry into the urethra, so that catheter 300 is not exposed to the outside atmosphere.

In this example embodiment, extension 312 approximates a hollow hemisphere open on both ends for purposes of catheter insertion. The proximal end of extension 312 is attached to the distal end of guide 310. The diameter of the proximal end of extension 312 matches or exceeds the diameter of an opening in guide 310. The diameter of extension 312 immediately enlarges from the proximal end of extension 312, then bevels inward toward the distal end of extension 312 to form its hemispherical shape. Using extension 312, as soon as sheath 302 starts to bunch up as catheter 300 is being inserted into the opening of guide 310, extension 312 prevents excess material of sheath 302 from getting pulled into guide 310. In this manner, extension 312 partitions sheath 302 and guide 310 making insertion much easier.

Other embodiments of the extension include many different forms and shapes suitable for preventing a sheath from getting pulled into a guide. Some embodiments feature an extension shaped for efficient manufacture while others are shaped for maximum prevention. These and other forms will become readily recognizable to those having skill in the art upon reading this disclosure.

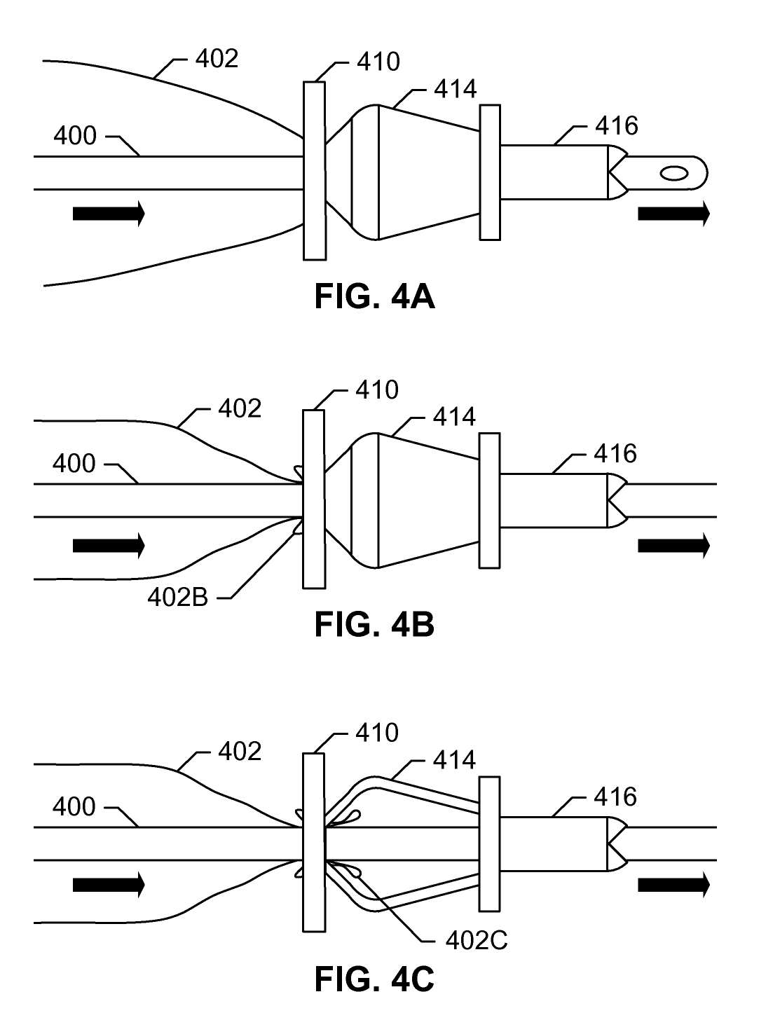

FIG. 4A shows a guide 410 of a catheter assembly without an extension during insertion. The catheter assembly includes a catheter 400, a sheath 402, a guide 410, a reservoir 414, and an introducer 416. A substantial portion of the length of catheter 400 is surrounded by and enclosed within sheath 402, which acts as a protective envelope for catheter 400, and converges at guide 410. The proximal end of catheter 400 slides through an opening in guide 410 to enter reservoir 414. However, there is no extension attached to the distal end of guide 410 to partition sheath 402 from guide 410. The proximal end of guide 410 is attached to reservoir 414, which contains a lubricant, water, or saline. As catheter 400 enters reservoir 414 through guide 410, catheter 400 is lubricated before urethral insertion. Introducer 416 is attached to the proximal end of reservoir 414. Introducer 416 is a hollow cylinder with a diameter just large enough to allow catheter 400 to slide smoothly through and into the urethra. Introducer 416 may enter the urethra prior to catheter entry into the urethra, so that catheter 400 is not exposed to the outside atmosphere.

In FIG. 4A, the initial stage of catheter insertion is shown. The arrows indicate the movement of the catheter through guide 410, reservoir 414, and introducer 416. The proximal end of catheter 400 has just gone through introducer 416. At this point, sheath 402 has not begun to bunch up, and has not begun to get pulled into guide 410.

FIG. 4B shows the bunching of sheath 402 during insertion. At this point, catheter 400 has traveled through guide 410, reservoir 414, and introducer 416 a little further along the direction of the arrows. Sheath 402 has begun to bunch up, and excess material 402B of sheath 402 has started to get pulled into guide 410.

FIG. 4C shows the inside of guide 410 during insertion. At this point, catheter 400 has traveled even further through guide 410, reservoir 414, and introducer 416 along the direction of the arrows. Sheath 402 has bunched up, with additional excess material 402C of sheath 402 being pulled into guide 410. Since guide 410 includes an opening having approximately the same diameter as catheter 400, having portion 402C within the opening of guide 410 as well makes it more difficult to push catheter 400 through guide 410, especially for users having limited manual dexterity. This difficulty can increase as more excess material of sheath 402 enters the opening of guide 410.

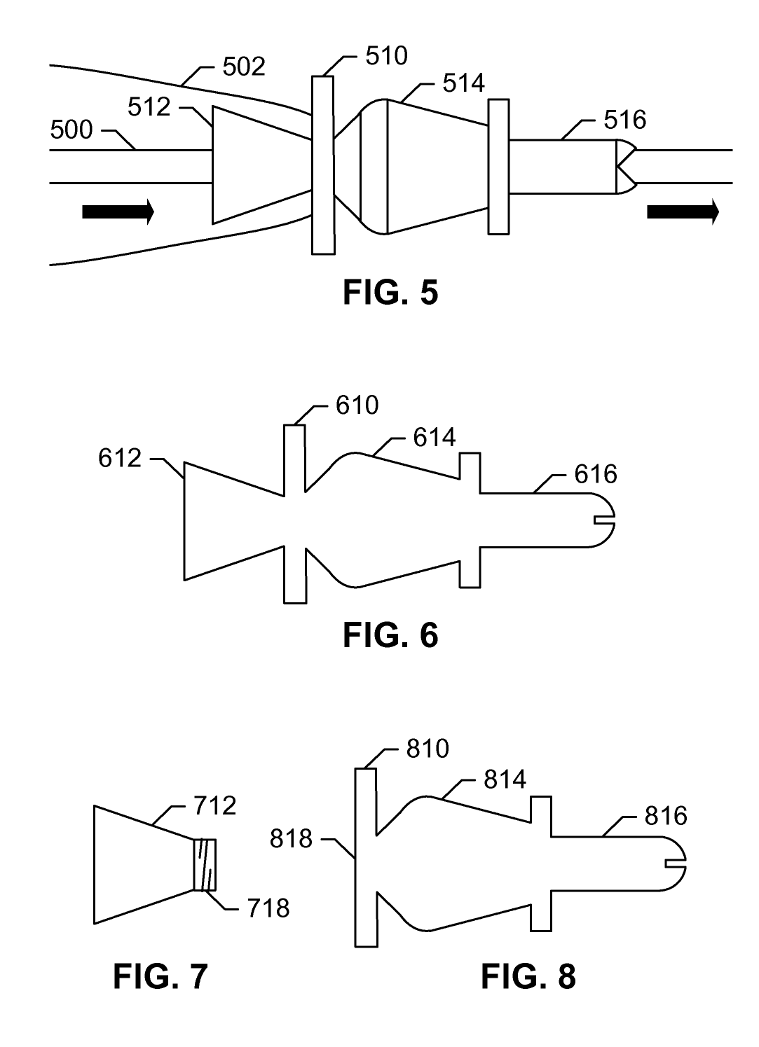

FIG. 5 shows a catheter assembly having an extension 512 during insertion, according to an example embodiment of the present subject disclosure. In this example embodiment, the catheter assembly includes a catheter 500, a sheath 502, a guide 510, extension 512, a reservoir 514, and an introducer 516. At this point, the catheter has traveled through the opening of guide 510, reservoir 514, and introducer 516. As the catheter is being inserted, sheath 502 is bunched up forming excess material, but has not been pulled into the opening of guide 510. Extension 512 forms a barrier, or partition between sheath 502 and guide 510, which prevents sheath 502 from getting pulled into guide 510.

FIG. 6 shows a guide 610 and an extension 612 as a single unit, according to an example embodiment of the present subject disclosure. In this example embodiment, guide 610, extension 612, reservoir 614, and introducer 616 are manufactured as a single unit. This single unit may include a throughbore through its length adapted for receiving a catheter. The manufacturing can be accomplished using thermoforming, injection molding, etc. Manufactured as a single unit, this unit can be attached to a sheath and catheter to complete a catheter assembly that operates as described herein. In other example embodiments, the single unit may not include a reservoir and/or an introducer, but continues to maintain the function of separating a sheath from a throughbore of the unit during insertion of a catheter.

FIG. 7 shows an extension 712 for attachment to a guide, according to an example embodiment of the present subject disclosure. In this example embodiment, extension 712 includes fastener 718. Fastener 718 is used to connect extension 712 to a guide. The guide may have a complementary fastener to strengthen the connection, or for efficient connection. Fastener 718 may be screw threads for screwing extension 712 into a guide as shown. Fastener 718 can also include snaps, or any other protrusion or indentation which facilitates connection with a guide. These and other connections will become readily recognizable by those having skill in the art upon reading this disclosure. In other example embodiments, extension 712 does not include fastener 718, but connects to a guide using an adhesive, glue, external fasteners, etc.

FIG. 8 shows a guide for attachment to an extension, according to an example embodiment of the present subject disclosure. In this example embodiment, guide 810 includes a reservoir 814, an introducer 816, and a complementary fastener 818. Guide 810 includes an opening adapted to receive a catheter, and is adapted to receive an extension for preventing a sheath from getting pulled into the opening of guide 810. For instance, complementary fastener 818 is used to connect an extension to guide 810. Complementary fastener 818 may be screw threads for screwing an extension into guide 810, etc. Complementary fastener 818 can also include snaps, or any other protrusion or indentation which facilitates connection with a guide. These and other connections will become readily recognizable by those having skill in the art upon reading this disclosure.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A urinary catheter assembly, the assembly comprising:
a catheter having a proximal end and a distal end;
a guide adapted to receive the proximal end of the catheter via an opening;
a sheath surrounding a substantial portion of the catheter, wherein a proximal end of the sheath is attached to the guide; and
a hollow extension attached to a distal end of the guide, the hollow extension being enclosed by the sheath and being placed in between the sheath and the opening.

2. The assembly of claim 1, wherein the extension has a greater distal diameter than a proximal diameter.

3. The assembly of claim 2, wherein the extension is in the form of a cone.

4. The assembly of claim 2, wherein the extension is in the form of a hemisphere.

5. The assembly of claim 1, wherein the extension maintains a substantially similar shape during catheter insertion.

6. The assembly of claim 1, wherein the extension and the guide are made from a substantially similar material.

7. The assembly of claim 1, wherein the proximal end of the guide is connected to a reservoir including a lubricant for lubricating the catheter as it moves through the reservoir.

8. An extension for a urinary catheter assembly, the extension comprising:
a hollow member; and
an attachment portion at a proximal end of the hollow member, the attachment portion attachable to a distal end of a guide adapted to receive a catheter;
wherein the hollow member is in between the catheter and a sheath substantially surrounding the catheter when attached to the distal end of the guide.

9. The extension of claim 8, wherein the attachment portion is adapted to removably attach to the distal end of the guide.

10. The extension of claim 8, wherein the attachment portion is adapted to fixedly attach to the distal end of the guide.

11. The extension of claim 8, wherein the hollow member has a distal diameter that is greater than a proximal diameter of the hollow member.

12. The extension of claim 11, wherein the hollow member is in the form of a cone.

13. The extension of claim 11, wherein the hollow member is in the form of a hemisphere.

14. The extension of claim 8, wherein the hollow member maintains a substantially similar shape during catheter insertion.

15. An apparatus for a urinary catheter assembly, the apparatus comprising:
a guide adapted to receive a proximal end of a catheter via an opening in the guide, and attachable to a proximal end of a sheath substantially surrounding the catheter; and
a hollow extension coupled to a distal end of the guide;
wherein the extension is situated in between the sheath and the opening.

16. The apparatus of claim 15, wherein the extension has a greater distal diameter than a proximal diameter.

17. The apparatus of claim 16, wherein the extension is in the form of a cone.

18. The apparatus of claim 16, wherein the extension is in the form of a hemisphere.

19. The apparatus of claim 15, wherein the extension maintains a substantially similar shape during catheter insertion.

20. The apparatus of claim 15, further comprising a reservoir coupled to the proximal end of the guide including a lubricant for lubricating the catheter as it moves through the reservoir.
